(19) United States
(12) Patent Application Publication
(10) Pub. No.: US 2005/0014146 A1
(43) Pub. Date: Jan. 20, 2005

(54) Method and device for integrated biomolecular analyses

(76) Inventors: Nicolo Manaresi, Bologna (IT); Gianni Medoro, Trinitapoli (IT); Luigi Altomare, Bologna (IT); Marco Tartagni, Meldola (IT); Roberto Guerrieri, Bologna (IT)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/486,229

(22) PCT Filed: Aug. 7, 2002
(86) PCT No.: PCT/IT02/00524

(30) Foreign Application Priority Data
Aug. 7, 2001 (IT) TO01 A 000801

(51) Int. Cl. C12Q 1/68; G01N 33/53; G01F 1/64; G01N 27/26; G01N 33/50

(52) U.S. Cl. 435/6; 435/7.1; 205/777.5

(57) ABSTRACT

A method whereby first biological entities are recognized by way of second biological entities able to bind to the first (or the first to the second), including the steps of binding first biological entities to a surface comprising an array of first electrodes selectively energizable and addressable at least in part, positioned facing at least one second electrode, bringing the second biological entities into contact with the first, these second biological entities and possibly the first being moved by means of dielectrophoretic cages generated between the electrodes, and sensing any binding activity between at least a portion of the first and of the second biological entities, preferably utilizing radiation at a first frequency to excite fluorophore groups bound to the second biological entities and detecting the emission of fluorescence at a second frequency by means of optical sensors integrated into the electrodes, the biological entities preferably being concentrated on the electrodes by the fusion of dielectrophoretic cages.

### TECHNICAL FIELD

The present invention relates to a method of molecular biological analysis utilizing dielectrophoretic forces to manipulate biological components advantageously and with high processivity. In particular, the method disclosed can be used to check the binding force between proteins and/or verify the presence and quantity of proteins in a sample, to assemble arrays of test points, to check the concentration of the proteins being tested, and, optionally, to observe the results with the aid of sensors integrated into the test device. The invention relates similarly to a device for implementation of the method thus outlined, equipped with the aforementioned integrated sensors.

### BACKGROUND ART

A great many immunological methods have been developed in recent years allowing the determination of antigens and antibodies, both for purely scientific and for diagnostic purposes.

Immunoassays

Immunological tests, or immunoassays, utilize a number of notably powerful methods for identifying and measuring antigens and antibodies. Specific antibodies are available for an increasing number of antigens, soluble, immobilized (on plates, resins or membranes), conjugated and otherwise. Moreover, with the range of systems for analyzing antigen-antibody complexes becoming steadily wider, and their sensitivity continuing to be improved, the potential and the range of applications for immunological reactions and techniques have been extended conspicuously. In the case of soluble antigens and antibodies, assays are based on the labelling of one of the reagents, on the formation and precipitation of immunocomplexes, or on the measurement of an effector function expressed by the antibody.

For some time, the most sensitive system available was radioimmunoassay (RIA), developed by Yarlow and Benson in 1960. This method betrays numerous drawbacks at all events, including the need for special equipment, also for special precautions against radiation (and for specially trained staff), and the limited average life of the radioactive isotopes used for labelling purposes. Such constraints soon led to the notion of replacing isotopes with enzymes as the labelling medium. The first studies on Enzyme Immunoassay (EIA) were conducted by Schuurs et al. and disclosed in a series of patents: U.S. Pat. Nos. 3,654,090; 3,791,932 and successive references. EIA methods include ELISA (Enzyme-Linked ImmunoadSorbent Assay) and its numerous variations, which currently are the methods of choice in the art fields of research and diagnostics. EIA-ELISA procedures are categorized as competitive and non-competitive, which in turn can be homogeneous or heterogeneous. Whilst homogeneous assays require no physical separation, heterogeneous assays require separation of the free antigen fraction from the fraction bound to the antibody, obtained by means of a solid phase system consisting generally in polystyrene, cellulose or nylon substrates to which the antibodies are bound. The substrates are usually 96- and 384-well microtiter plates or microstrips having 8, 12 and 16 wells, though they can also consist in single elements known as microbeads, on which the antigens or antibodies are immobilized. Competitive enzyme immunoassays are those where the antibody is present in a limited concentration. In non-competitive or immunometric assays, on the other hand, a notable excess of the antibody is used, conjugated with the enzyme, so as to maximize the antigen signal. Among non-competitive enzymatic immunoassays, the system most widely adopted involves capturing antigens from the sample on the walls of microsites coated with antibodies, generally monoclonal (mAb). The captured antigen is marked by coating it with a second layer of specific antibodies (secondary antibodies) with or without further amplification steps. The secondary antibody is often conjugated with an enzyme, the conversion of the enzyme demonstrating the presence of a given antigen: this is known as a sandwich ELISA assay.

With a wide range of substrates available for marker enzymes, it is possible to choose between different detection methods. The substrates are reagents that allow of displaying, qualifying and/or quantifying an analyte of interest in an enzyme immunoassay. Substrates can be chromogenic, chemiluminescent or fluorescent. Chromogenic substrates produce a coloured compound that can be identified visually and quantified with a spectrophotometer. Chemiluminescent substrates produce light that can be measured with a luminometer or recorded permanently on X-ray film. Fluorescent substrates on the other hand emit fluorescence that is measured with a fluorometer. Chromogenic and chemiluminescent substrates are excellent media for the detection of conjugates labelled with enzymes bound indirectly to a solid support. The enzymes commonly used for the purpose are peroxidase, generally Horse Radish Peroxidase (HRP), which catalyzes the fission of H₂O₂₁ Alkaline Phosphatase (AP), which removes the phosphate from phosphorylate molecules, and β-galactosidase (β-Gal), which hydrolyzes lactose. The conversion of numerous substrate molecules by a single enzyme molecule produces a notable amplification of the signal, though if a luminogenic or fluorogenic substrate is used, the signal/mass is still greater, comparable to that obtained with Radioimmunoassays.

EIA methods are powerful, but affected by the serious limitation of low productivity (given the difficulty of conducting significant numbers of analyses in parallel), due mainly to the scant possibilities for integration afforded by the various items of equipment needed to carry out the procedure. This makes it all but impossible to process thousands of samples simultaneously or at least in a short time, whereas speed is becoming more and more a fundamental aspect of modern research and diagnostics. In addition, EIA can involve a relatively heavy consumption of costly reagents.

Labelled Microbeads

Not least in order to overcome the aforementioned drawbacks, the use of microbeads labelled selectively employing various fluorescence methods is gaining more and more importance in the art field of biotechnologies. Especially pertinent in this field are the following patents:
WO 00/68692 in the name of Quantum Dot Corporation, which discloses various assay methods utilizing semiconductor nanocrystals, each emitting at distinct wavelengths, as specific markers for different microbeads;
WO 01/13120 A1 in the name of Luminex Corporation, which discloses microparticles emitting multiple fluorescence signals and methods for their use in a cytofluorometric system.

Molecular Sensors Based on Surface Plasmon Resonance

U.S. Pat. No. 5,641,640 in the name of BIAcore AB, discloses a system for the analysis of biological samples using surface plasmon resonance. Molecules of a sample held in suspension are directed into a chamber, of which the surface carries immobilized molecules potentially capable of binding with those of the sample. The binding of the molecules is sensed by indirect measurement of the variation in the refraction index caused by the binding of the molecules with the surface, observing the reflection from the surface of a suitable light source.

Dielectrophoresis

Dielectrophoresis relates to the physical phenomenon whereby dielectric particles subject to spatially non-uniform d.c. and/or a.c. electric fields undergo a net force directed toward those regions of space characterized by increasing (pDEP) or decreasing (nDEP) field strength. If the strength of the resulting forces is comparable to the force of gravity, it is possible in essence to create an equilibrium of forces enabling the levitation of small particles. The strength, direction and orientation of the dielectrophoretic force are heavily dependent on the dielectric and conductive properties of the body and of the medium in which it is immersed, and these properties in turn vary with frequency.

Studies analyzing the effects of dielectrophoretic forces on particles (the term “particles” is used hereinafter to indicate dielectrophoretically manipulated bodies or elements) consisting in biological entities (the term “biological entities” is used hereinafter to indicate cells and microorganisms, or parts thereof, namely DNA, proteins, etc.) or artificial objects consisting of inorganic matter, have suggested for some time the notion of exploiting these forces as a means of selecting a particular body from a sample containing a plurality of microorganisms, characterizing the physical properties of microorganisms and in general allowing their manipulation. Accordingly, it has been found advantageous to utilize systems comparable in size to those of the microorganisms being manipulated, and thus reduce the magnitude of the voltages used to create the field distributions needed to reveal the aforementioned effects.

Particles exposed to the phenomenon of dielectrophoresis are subject to forces dependent on the volume of the particle; this being the case, it has been assumed for some time that there must be a lower limit for particle size, beneath which dielectrophoretic force would be defeated by Brownian movement. It was considered that there would be a need for electric fields of magnitude such that local warming of the fluid would increase local flow and effectively prevent dielectrophoretic manipulation. Pohl (1978) speculated that the electric fields needed to trap particles smaller than 500 nm subject to Brownian movement would be too strong. The first group to overcome this obstacle was that of Washizu (Washizu et al., Trans. Ind. Appl. 30:835-843, 1994), who used positive dielectrophoresis to precipitate small proteins down to 25 kDa. This lowering of the threshold was favoured by improvements in electrode manufacturing technologies, notably the use of electron beams in manufacture. Thereafter, Fuhr et al. (Fuhr, 1995, Proc. St Andrews Meeting of Society for Experimental Biology p.77; Mueller et al., 1996, J. Phys. D: Appl. Phys. vol.29:340-349) and Green et al. (Green et al., 1995, Proc. St Andrews Meeting of Society for Experimental Biology p.77; Green et al., 1997, J. Biochem. Biophys. Methods vol.35:89-102) demonstrated that viruses of 100 nm diameter could be manipulated employing negative dielectrophoresis. It was also shown that latex microbeads of 14 nm diameter could be trapped both with positive and with negative dielectrophoresis (Mueller et al., 1996, J. Phys. D: Appl. Phys. vol.29:340-349). Subsequent studies showed that 68 kDa molecules of the protein avidin can be concentrated from solution using both positive and negative dielectrophoresis (Bakewell et al., 1998, Proc. 20th Ann. Int. Conf. IEEE Eng. Med. Biol. Soc. 20, 1079-1082).

Patent application PCT/WO 00/47322 discloses an apparatus and a method for manipulating particles utilizing closed dielectrophoretic potential cages, generated by singly and selectively addressable and mutually energizable adjacent electrodes making up an array.

Patent application PCT/WO 00/69565, filed by the same applicant, discloses a more efficient apparatus than that mentioned above and describes various methods of manipulating particles utilizing closed dielectrophoretic potential cages. The device described in this second PCT application is illustrated in FIG. 1 and comprises two basic modules; the first such module consists in a regularly distributed array. M of electrodes LIJ arranged on an insulating support (O1 in FIG. 1). The electrodes LIJ can be of any given conductive material, preference being given to metals compatible with electronic integration technology, whereas the insulating medium O1 can be silicon oxide or any other insulating material.

The electrodes of the array can be of any given shape; in the example of FIG. 1, the electrodes are square. Each element of the array M1 consists in an electrode LIJ that is selectively addressable and energizable in such a way as to generate a dielectrophoretic cage S1 (FIG. 1) by means of which to manipulate a particle, generally a biological entity (BIO in FIG. 1), all of which occurring in a liquid or semi-liquid environment denoted L in FIG. 1.

The region beneath the electrodes (C in FIG. 1) can be occupied by sensing means, and more exactly integrated circuits incorporating sensors of various types, able to detect the presence of single particles in potential cages generated by the electrodes.

In a preferred embodiment, the second main module appears substantially as a single large electrode M2, covering the device in its entirety. Finally, the device may also include an upper support structure (O2 in FIG. 1). The simplest form for the second electrode M2 is that of a plain flat and uniform surface; other forms of greater or lesser complexity are possible (for example a grid of given mesh size through which light is able to pass).

The most suitable material for the upper electrode M2 will be a transparent conductive material. Besides allowing the inclusion of sensing circuits as outlined previously, this will also allow the use of traditional optical inspection means (microscope and TV camera) located above the device.

Among the singular aspects of the invention disclosed in patent application PCT PCT/WO 00/69565, parts of which are incorporated into the present specification where necessary for reference purposes, is that the one substrate can accommodate both the elements capable of manipulating the particles (biological entities), and the sensing devices.

### DISCLOSURE OF INVENTION

The object of the present invention is to overcome the drawbacks inherent in the prior art methods outlined above for conducting biomolecular tests on biological entities (cells, microorganisms or parts thereof, in particular oligonucleotides, proteins or parts thereof) in such a way that these tests can be carried out swiftly, efficiently and economically, with precision and high processivity, using smaller quantities of reagents and especially of costly reagents, namely monospecific antibodies, labelled antibodies and substrates.

Here and in the following description, the term “protein” is used to indicate a molecular chain of amino acids bound by peptide bonds; the term does not refer to a specific length, and accordingly, the commonly used terms “polypeptide”, “peptide” and “oligopeptide” are also included in the definition. Also included are post-translational modifications of protein such as glycosilations, acetylations, phosphorylations and the like. Moreover, the term protein likewise includes protein fragments, analogues, mutated or variant proteins, fusion proteins, and so forth.

Just as the term antibody can be taken, where not explicitly stated, to mean antibodies obtained from polyclonal and/or monoclonal preparations, it can also be taken to mean chimeric antibodies, F(ab′)2 and F(ab) fragments, Fv molecules including single chain (sFv), dimeric and trimeric constructs of antibody fragments and any fragment obtained from these and similar molecules, where these happen to maintain the specific binding properties of the original antibody molecule.

In the light of the foregoing definitions, one object of the present invention in particular is to exploit the potential afforded by the device of patent application PCT/WO 00/69565 in providing a method of conducting integrated biomolecular analysis on a biological sample including unknown biological entities, for example specific proteins or antigens or specific antibodies, by means of known biological entities, typically antibodies, or natural or synthetic proteins, such as can be run with a high level of automation and in parallel, if necessary, on a high number of samples, or on a significant number of different biological entities in one sample.

The stated objects are realized in a method according to the present invention for conducting integrated biomolecular analyses on a biological sample including unknown biological entities, with the aid of known biological entities capable of binding to the unknown biological entities, comprising the steps of immobilizing first biological entities directly or indirectly on a support, bringing second biological entities into contact with the first and detecting any binding activity between at least a proportion of the first biological entities and at least a proportion of the second biological entities; the first or second biological entities being the unknown entities and the second or first biological entities being the known entities; characterized:
(A)—in that the support is provided by a surface consisting in an array of first electrodes, selectively energizable and addressable at least in part, disposed facing and distanced by means of a spacer from at least one second electrode, in such a manner that the second electrode, the spacer and the array of first electrodes combine to establish a test chamber such as will compass a liquid or semi-liquid environment in which closed dielectrophoretic cages are generated selectively by means of the first electrodes and the second electrode, for the purpose of trapping and moving at least the second biological entities in the chamber; and,
(B)—in that the surface is treated beforehand in such a way as to promote binding with the first biological entities at the first electrodes. In particular, the immobilizing step comprises the single steps of:
a. introducing a suspension of the first biological entities into the chamber compassing the liquid or semi-liquid environment;
b. trapping and levitating the first biological entities within dielectrophoretic potential cages generated between selected first electrodes and the second electrode;
c. selectively directing the dielectrophoretic cages, with the first biological entities trapped inside them, toward selected first electrodes;
d. moving the cages in such a way as to promote binding between the first biological entities and the selected first electrodes, and consequently immobilizing the first biological entities on the electrodes, according to a predetermined patterning sequence.

One of the singular features of the method according to the invention consists moreover in the facility of concentrating antigens and/or antibodies involved in the analysis by attracting them into the dielectrophoretic cages. Other characterizing features of the method disclosed include the facility of generating protein microarrays, by dielectrophoretic manipulation of the protein population of interest, which can then be assayed to reveal their affinity with other proteins (antigens or antibodies). Moreover, the specificity of the antigen-antibody bond can be tested electronically by trying to separate the bound proteins, seeking to draw one of them back into the dielectrophoretic cages by varying the particular force and/or frequency of the cage. The test can be monitored exploiting standard methods (fluorescence, luminescence or colour development) and employing optical sensors, which can be external (microscope and TV camera) or integrated into the device. Alternatively, it is possible to use a method exploiting capacitive sensors integrated into the device to observe the formation of antigen-antibody complexes.

A further object of the invention is to provide a device for conducting molecular biological analyses that will be notably compact, economical and reliable, while capable of fully automated operation and processing at high speed.

The stated object is realized according to the present invention in a device for molecular biological analyses performed with the aid of movable dielectrophoretic cages, comprising a surface afforded by an array of first electrodes selectively energizable and addressable at least in part and arranged on an insulating support; at least one second electrode positioned opposite and facing at least a part of the array of first electrodes; and a spacer serving to distance the first electrodes from the at least one second electrode in such a way that the second electrode, the spacer and the array of first electrodes combine to establish a test chamber encompassing a liquid or semi-liquid environment; characterized in that it further comprises integrated optical sensors located beneath or in close proximity to at least one of the first electrodes; and in that the first electrodes comprise means by which to allow the transmission of electromagnetic radiation through the selfsame first electrodes and toward the optical sensors, operating in conjunction with means likewise forming part of the device and positioned to coincide with the first electrodes, by which radiation of a first predetermined wavelength is prevented from reaching the integrated optical sensors.

The advantages of the present invention are many and various.

The proposed method guarantees high sensitivity thanks to the possibility of concentrating the protein populations present in samples by attracting them selectively into the dielectrophoretic cages. This naturally signifies a saving in expenditure on reagents, as well as the facility of testing samples to the limit of the detection potential afforded by standard methods.

Another singular advantage is the facility of verifying the specificity of the assay by way of an electronic antigen-antibody binding affinity check, which will eliminate false positives generated by possible cross-reactivity of the antibodies, a likelihood that cannot be excluded when handling thousands of antigens or antibodies together. This procedure also allows the stability of the antigen-antibody bond to be evaluated directly.

Complementing the high sensitivity obtainable with the method according to the present invention is an appreciable parallelism, given that the assay can be conducted on all the proteins in a single chamber rather than in a plurality of distinct, albeit very similar chambers. This, together with the high level of integration and feedback control achievable thanks to the automation allowed by the device and the method disclosed, means that any variability of response given by the assay due to system-related and/or accidental (operator) errors can be reduced to a minimum. Another advantage of the method is that of integrated sensing, which dispenses with the need for cumbersome instruments (fluorometers, luminometers, etc.), which very often are not even associated with the test device. In the case of direct capacitive sensing, the experimenter avoids the need for labelling of the antibodies employing generally complex and costly procedures, to facilitate their identification. Likewise in the case of capacitive (indirect) labelling by means of microbeads, the procedure is particularly simple and applicable even to antigenic proteins.

In the case of a directly assembled protein array, exceptionally high density is achievable given that thousands of different proteins can be patterned on the electrodes of the device, which are spaced at a particularly fine pitch.

Other features and advantages of the invention will emerge more clearly from the following description of certain preferred embodiments illustrated by way of example, and implying no limitation, with the aid of the accompanying drawings.

### BRIEF DESCRIPTION OF THE DRAWINGS

FIG. 1 is a schematic three-dimensional view showing part of a prior art device for the manipulation of a sample, which presents a modular structure composed of a support containing the electrodes, and a lid;

FIG. 2 illustrates one possible embodiment of an integrated optical sensor according to the present invention;

FIG. 3 is a detailed step-by-step illustration of the method according to the invention;

FIG. 4 illustrates a test procedure in which the sample containing the protein to be identified is immobilized on the electrodes, whereupon dielectrophoretic cages are generated above the electrodes;

FIG. 5 shows another way of conducting an immunological assay according to the invention, in which there is no need to move the cages;

FIG. 6 shows the spectral emission response of certain fluorescent molecules excited by a monochrome laser source emitting ultraviolet radiation at 405 nm;

FIG. 7 illustrates an enlarged detail of FIG. 2, viewed schematically and representing a cross section through a planar MOS device associated with a well diffusion;

FIG. 8 shows the spectral responses, calculated mathematically on the basis of the semiconductor device equations, interpolated with silicon related experimental absorption data, of the two junctions of FIG. 7 for a typical CMOS device with detail definition of 0.7 μm.

### BEST MODE FOR CARRYING OUT THE INVENTION

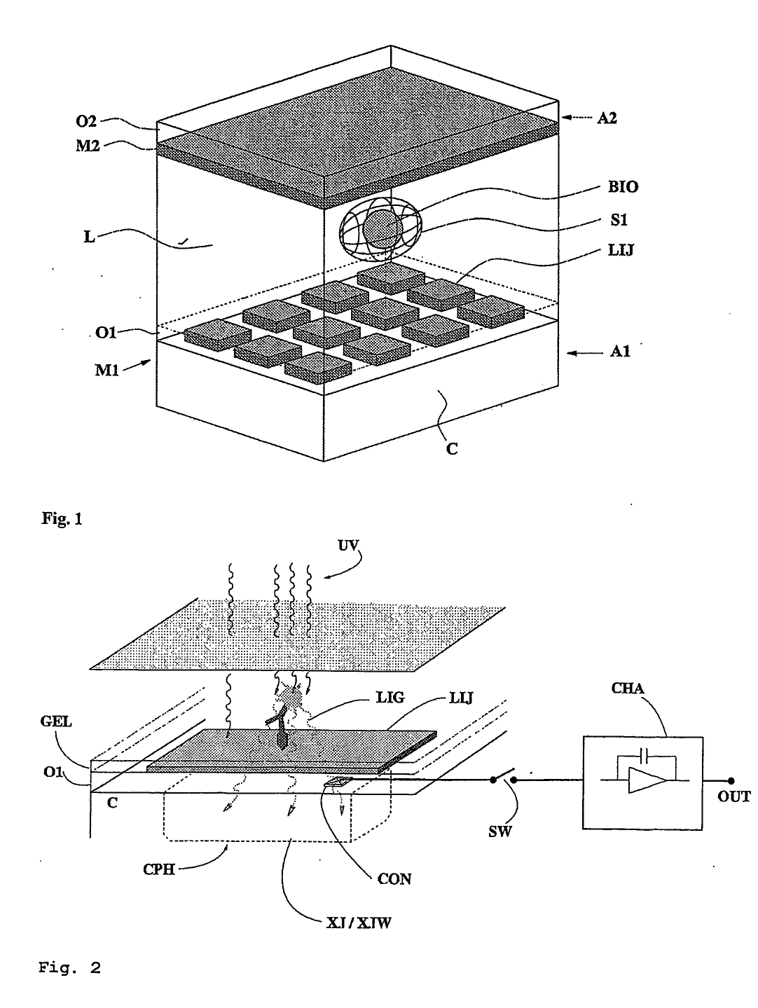

With reference to FIGS. 1 and 2, the device disclosed in patent application PCT WO/00/69565 (or a similar prior art device) is equipped according to the present invention with optical sensors capable of indicating the presence or absence of a biological element suspended in buffer solution within a dielectrophoretic cage. The electrode LIJ affords an opening, or window, of dimensions such as will not significantly affect the dielectrophoretic potential generated but nonetheless allow the passage of a certain amount of light radiation coming from a source external to device. The lid A1 is conventional in embodiment, fashioned from a semi-transparent conductive material in such a way that the transmission of the light radiation will not be impeded. The space beneath the window in the electrode LIJ is occupied by a silicon substrate C and, conventionally, a charge-storage junction photodiode CPH. The presence or absence of the biological element BIO will influence the amount of light radiation incident on the photodiode, thus varying the quantity of charge accumulated over the integration time. The variations induced in stored charge status are revealed by a conventional charge amplifier CHA composed of: an operational amplifier, a feedback capacitor and a reference voltage VRE. The connection with the charge amplifier is obtained by enabling a suitable switch SW1, which might be located in the electrode LIJ. The photodiode and the charge amplifier are designed, applying prior art principles, to give a signal/noise ratio sufficient to verify the presence or absence of the biological particle.

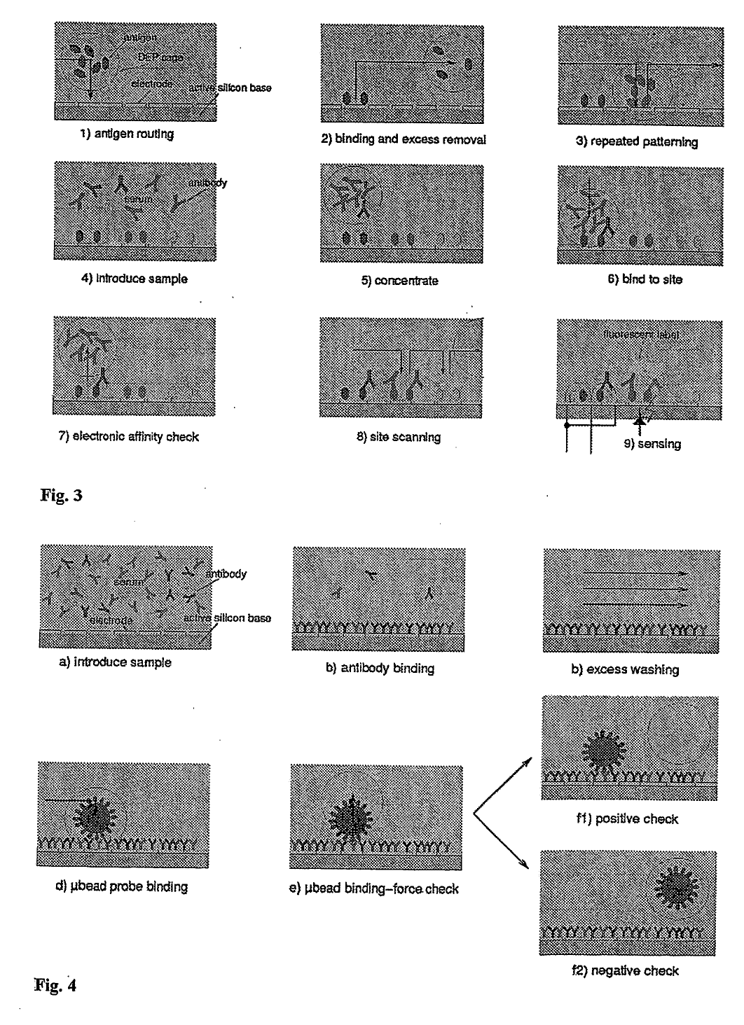

The method according to the present invention is carried into effect, unless otherwise indicated, employing conventional chemical and biochemical procedures commonly used and widely documented in literature. The preferred procedure, though not exclusive and implying no limitation whatever, is that illustrated in FIG. 3.

The procedure begins with construction of the protein array to be tested; the array in the example of FIG. 3 is composed of antigen proteins, though these might equally well be antibody proteins. The sample containing a homogeneous population of antigen proteins that will constitute the first element of the array is introduced into the device, and more exactly into the environment denoted L. The population is concentrated by attracting the molecules into a single dielectrophoretic cage. When the cage is moved, the antigen population trapped in the cage will move also, and this dielectrophoretic manipulation facility is used to route the antigens onto a selected electrode LIJ, which may be suitably functionalized (FIG. 3, step 1); in any event, the surface afforded by the array of electrodes LIJ will have been treated beforehand in a conventional manner so as to promote binding with the biological entities, in this instance antigens, at the selfsame electrodes LIJ. Lowering the dielectrophoretic cage, or deactivating the cage and exploiting diffusion, part of the molecules will bind to the electrode, through the agency of functionalized groups if included, whilst the unbound molecules are removed by raising or reactivating the cage and distancing it from the selected electrode (FIG. 3, step 2). This patterning step is repeated sequentially for all the antigens in the array (FIG. 3, step 3).

Alternatively, the protein array to be tested on the electrodes can be prepared using standard microarray technology, such as ink-jet.

At this point the sample containing the biological entities to be tested (mixture of antibodies) is introduced into the device (FIG. 3, step 4). The antibody population can be concentrated by attracting the molecules into a single dielectrophoretic cage (FIG. 3, step 5). The cage is manipulated in such a way as to offer the trapped antibodies to the first site (selected electrode LIJ or neighbourhood) where there are antigens present (FIG. 3, step 6). Thereupon, any antibodies in the sample that may be specific to the antigen bound to the site will now bind in their turn to the antigen, thereby confirming the presence of antibody proteins present in the sample, and conceivably the quantity. Next, the cage is distanced from the site (FIG. 3, step 7), possibly varying the parameters (field strength, frequency) to vary the dielectrophoretic force, in such a way as to remove the non-specific antibodies and at the same time verify the specificity of any antigen-antibody bonds. The procedure is repeated for all of the sites making up the array (FIG. 3, step 8).

The test can be monitored exploiting methods that use fluorescence, chemiluminescence, etc. In the example of the drawing, an antibody population is labelled with a fluorescent marker molecule (FIG. 3, step 9) detectable with optical sensors that can be stationed externally to the test chamber compassing the test environment L (microscope, TV camera), or integrated into the device, and more particularly into the substrate C beneath the array of electrodes LIJ. In this instance it is the antigen protein immobilized on the electrode that is identified by means of the labelled antibody.

An alternative option would be to use a method exploiting capacitive sensors integrated into the device (conventional in embodiment and therefore not illustrated), such as will indicate the capacitance associated with the electrode of each single protein site established previously and show the difference in capacitance when another protein binds to those already present at the site (FIG. 3, step 9). Utilizing this system, the protein to be identified can be either the protein bound to the surface of the electrodes LIJ or the protein soluble in the liquid or semi-liquid environment L, whether antigen or antibody. To this end, the dielectric characteristics of the proteins that serve to bring about recognition, be they antigen or antibody, can be modified by immobilizing them on microsupports, for example microbeads of a synthetic material that might have known physical characteristics (colour, fluorescence, etc.), in addition to their particular dielectric constant, such as will facilitate recognition internally of the device. In this instance the method according to the invention will also include a step of recognizing the microbeads, conducted according to the nature of these physical characteristics.

The variation in capacitance can be identified employing the methods and circuits disclosed in patent application PCT/WO 00/69565.

One variation on the method according to the present invention relates to a test procedure in which the sample containing the proteins to be identified is immobilized in spatially uniform manner on the surface of the device, above the electrodes, as indicated schematically in FIG. 4. In this version of the method, a biological sample (serum) containing an unknown heterogeneous antibody population (FIG. 4, step a) is introduced into the device. The antibodies bind to the electrodes, which can be passivated and/or suitably functionalized (FIG. 4, step b). Any excess of unbound antibodies is removed by flushing buffer solution through the chamber of the device (FIG. 4, step c). Probe microbeads are then introduced into the device, each coated with a known protein that could bind one of the antibodies. The microbeads are manipulated dielectrophoretically and brought directly into contact with the antibodies covering the electrodes. Alternatively, contact with the antibodies can be brought about by manipulating the microbeads onto the vertical axes of the electrodes, likewise dielectrophoretically, then deactivating the cages (gravitational method). Binding activity is verified by seeking to raise the dielectrophoretic cage or, alternatively, simply reactivating it in the event that the microbead was deposited gravitationally. The sensing procedure consists in measuring the difference in capacitance between the electrode and the bead in contact with it or raised in the cage, or moving the cage further, between the electrode with a bead bound and another one with no beads bound. The presence of a suspected antibody and, if envisaged, an estimate of its concentration, is verified by assessing the number of microbeads bound.

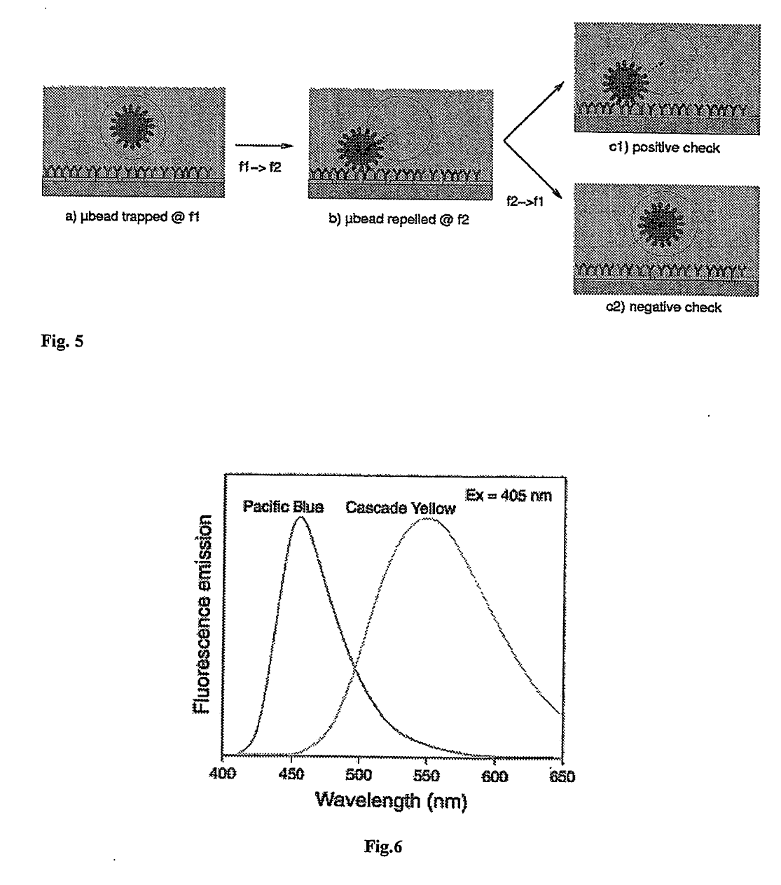

FIG. 5 illustrates another procedure suitable for running the same test. In this instance there is no need for movement of the cages and therefore the method can be implemented using a less complex device, in which the additional circuitry consists in nothing more than the capacitive sensing circuit. This version of the method disclosed exploits the change in dielectrophoretic force from negative (nDEP) to positive (pDEP). In step a) of FIG. 5, the microbead, functionalized with protein, is trapped at a given frequency f1 in a potential cage above the electrodes (negative dielectrophoresis). Changing to frequency f2 and increasing the field strength (pDEP) the bead is repelled by the cage and attracted toward a maximum potential, i.e. onto the electrodes, where it enters into contact with the immobilized antibodies (FIG. 5, step b).

The antibody-protein binding check is run simply by resetting the frequency to f1; if binding has occurred, the microbead will not be able to return inside the cage (FIG. 5, step c1), whereas if binding has not occurred, the cage will again be able to attract the microbead (FIG. 5, step c2).

Clearly, the microsupport selected for immobilization of the biological entities to be manipulated and/or identified can be a medium other than a microbead; for example, the molecules of interest might be immobilized on the surfaces of cells or liposomes.

In accordance with a further variation on the method, moreover, the antigen-antibody binding force check can be run without using dielectrophoresis, but simply introducing a flow of buffer solution into the environment L, directed through the surrounding chamber; in this instance it will be hydrodynamic force that induces the bound biological entities to separate from the surface afforded by the electrodes LIJ.

To enable the detection of fluorescent marker molecules, whether associated directly with the biological entities or with microbeads (or with other microsupports as mentioned above), the device of FIG. 1 is exposed to electromagnetic radiation at a first predetermined wavelength, for example ultraviolet UV (FIG. 2) falling directly on the samples BIO occupying the environment L compassed by the chamber. The elements labelled with fluorescent molecules are selected in such a way, accordingly, as to emit electromagnetic radiation at a second predetermined wavelength different to the first, for example in the visible spectrum; this radiation can be detected advantageously by sensors integrated into the silicon substrate C. By way of example, FIG. 6 shows the spectral response for emission from certain typical fluorescent molecules excited with a monochrome laser emitting ultraviolet radiation at 405 nm.

In accordance with the state of the art, the typical excitation wavelengths for these molecules range from 350 to 480 nm for Ar, Xe—F and Xe ion lasers. It is therefore important that the optical sensors incorporated into the substrate C should be selective, in particular, not liable to react to ultraviolet radiation, and especially sensitive to radiation in the visible spectrum. This performance potential can be delivered by employing suitable techniques for the embodiment of semiconductor type optical sensors, which also constitute subject matter of the present invention, as will now be explained.

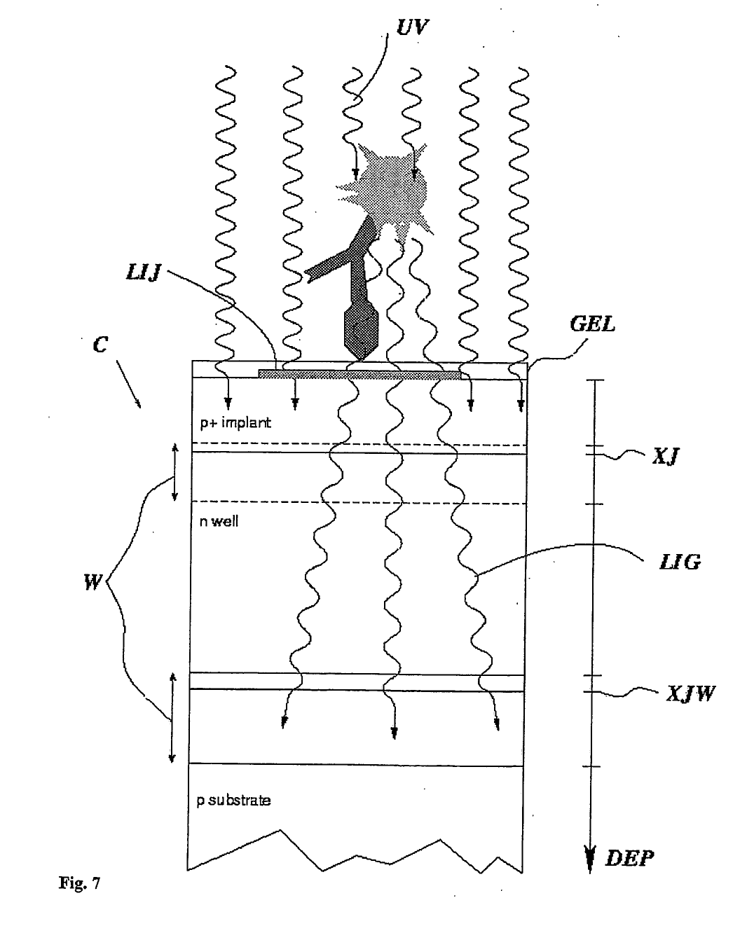

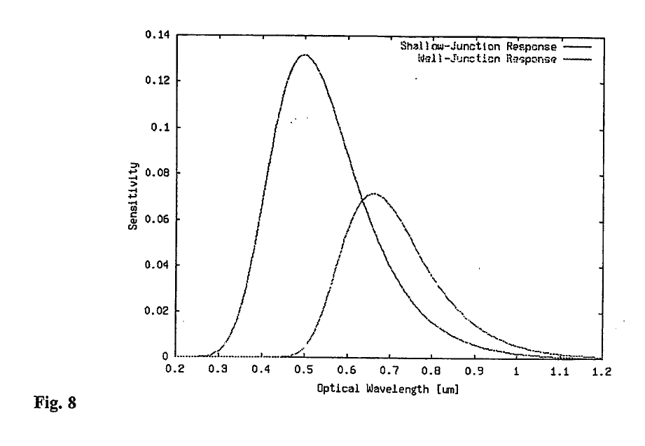

In general, a photon related to the ligh flux LIG (FIGS. 2 and 7) penetrates the substrate C of a semiconductor to the point at which, interacting with a crystal lattice, it pushes an electron from the valence band to the conduction band, in other words generating an electron-hole pair. The probability with which this phenomenon occurs depends on the average depth to which the photon penetrates the substrate and is directly proportional to its energy. The energy of the photon is E=h c/λ (where h=Planck constant, c=speed of light), hence a function of wavelength λ, and therefore the probability of generation is closely related to this latter quantity. Generally considered, experimental data obtainable on silicon substrates show a high generation probability for wavelengths of the order of 200-300 nm, which reduces markedly and exponentially at wavelengths of 800-1000 nm. This phenomenon translates into the fact that photon flux is characterized by a mean penetration length into the silicon dependent on wavelength: a few tens of micrometres (millionths of one metre) for emissions in the ultraviolet range, and several micrometres for those in the infrared range.

One method commonly utilized to quantify photogenerated charges, and thus measure the intensity of the photon stream, consists in establishing a reverse biased p-n junction (XJ or XJW) in the region through which the flux is directed. A device embodied in this fashion is known as a photodiode, denoted CPH in FIG. 2. The charges generated by light in the space-charge region W are drawn to the boundaries of this same region by the strong electric field and are quantifiable: 1) by measuring the current they generate, having biased the junction at constant voltage; 2) by measuring the total charge accumulated at the end of a set time during which the photodiode is not biased (storage-mode technique).

Utilizing planar technology, the foregoing operations are implemented according to the present invention by placing a contact CON on the diffusion surface of the photodiode CPH, such as can be connected electrically by way of an electronic address switch SW to the input of an electronic charge amplifier CHA. The output OUT of the charge amplifier encodes the amount of charge and therefore the luminous intensity incident on the photodiode CPH. It is possible to demonstrate that the space-charge region is the main factor responsible for photogeneration current.

The response of the photodiode as a function of the wavelength of the incident radiation thus depends to a considerable extent on the depth DEP of the junction (FIG. 7): on the one hand, radiation of short wavelength (ultraviolet) is absorbed in the immediate neighbourhood of the surface, in this instance not penetrating the space-charge region W, whereas on the other, radiation of relatively long wavelength and bordering on the visible (infrared) will penetrate further into the space-charge region, though with less likelihood of photogeneration occurring. By reason of these two opposite types of behaviour, peak sensitivity of the photodiode will be localized in the region of the visible, with minimal sensitivity registering at wavelengths in the infrared and ultraviolet range. Thus, the photodiode embodied in accordance with the present invention has a sensitivity to different types of radiation as characterized by the humped curves of FIG. 8, with peak sensitivity tending to register at wavelengths in the infrared spectrum for deeper junctions.

Current MOS planar technology affords different possibilities for the manufacture of photodiodes: in particular, the preferred solution consists in diffusion using shallow-junctions and well-junctions. FIG. 7 illustrates a cross section through a planar MOS device at a well diffusion. More exactly, the drawing shows shallow junctions XJ and well junctions XJW. FIG. 8 shows the spectral responses of the two junctions, calculated mathematically on the basis of the semiconductor device equations, interpolated with experimental absorption data relative to silicon, for a typical CMOS photodiode with detail definition of 0.7 μm. The depths DEP of the two junctions are 0.28 μm for the shallow (XJ) and 2.7 μm for the well (XJW). In accordance with what has already been stated, the spectral response of the deeper junctions, notably the well, indicates a marked sensitivity to infrared radiation and minimal sensitivity to ultraviolet.

In conclusion, the use of a deep well junction is particularly suitable for the proposed application, in order to eliminate the influence of ultraviolet radiation while maintaining good sensitivity at visible wavelengths.

Another way of increasing the selectivity of the sensors or more simply ensuring a higher level of confidence when using surface junctions (such as those deriving from the most sophisticated technologies), obtainable following procedures already familiar in the art field of semiconductor device manufacture, is that of utilizing suitable colour filters GEL deposited on the surface of the substrate C. These filters can be overlaid on the chip by means of photolithography and consist in colour photoresists or gels characterized by deposition resolutions of a few tenths of one micrometre (μm). In the example of the present disclosure, any ultraviolet interference can be reduced by using filters tuned in the yellow or green colour range.

In one possible embodiment of optical sensing means according to the invention, the p-n junction XJ or XJW is located in the silicon region C beneath the electrode LIJ, the electrode being fashioned photolithographicaly from materials that are electrically conductive, but transparent, typically Indium Tin Oxide (ITO). This solution can be obtained by post-processing an integrated circuit produced using the standard silicon technology applied routinely in microelectronics manufacturing processes, whereby the final passivation layer is applied in such a way as to leave portions of the metallization raised and exposed. The metallization is then used to establish an electrical contact between the transparent electrode and the circuits beneath.

In other solutions, utilizing an electrode LIJ of conventional embodiment that may not be transparent to light radiation, the photodiode could be located in the substrate, occupying the gap between the single electrodes, and the signals selected in such a way as to position the potential cage exactly in the space above the gap. In a further possible solution, electrodes embodied in non-transparent material could be fashioned with a central window, as mentioned previously, through which light can be directed so as to fall on the substrate beneath incorporating a photodiode.

Lastly, another way of preventing radiation emitted at the first frequency (UV in the example illustrated) from falling on the photodiode, is to create a waveguide utilizing the oxide of the chip and the glass of the lid, which will allow the fluorophores in the sample to be excited by radiation at a first frequency, directed laterally into the chamber holding the sample. The waveguide created in this manner will prevent the excitation energy from penetrating the substrate, since the unwanted radiation is reflected from the surface of the array by reason of its minimal angle of incidence, whilst that emitted by the fluorophores at given points of the array, being omnidirectional, will penetrate the surface of the array.

1-31. (Canceled)

32. A method of conducting integrated biomolecular analyses on a biological sample including unknown biological entities, with the aid of known biological entities capable of binding to the unknown biological entities, comprising the steps of immobilizing first biological entities directly or indirectly on a support, bringing second biological entities into contact with said first biological entities and detecting any binding activity between at least a proportion of said first biological entities and at least a proportion of said second biological entities; said first or said second biological entities being said unknown entities and said second or said first biological entities being said known entities; characterized:
(A)—in that said support is provided by a surface consisting in an array of first electrodes (LIJ) selectively energizable and addressable at least in part, disposed facing and distanced by means of a spacer from at least one second electrode (M2), in such a manner that said second electrode, said spacer and said array of first electrodes (LIJ) combine to establish a test chamber such as will compass a liquid or semi-liquid environment (L) in which closed dielectrophoretic cages (S1) are generated selectively by means of said first electrodes (LIJ) and said second electrode (M2) for the purpose of trapping and moving at least said second biological entities in said chamber;
(B)—in that said surface is treated beforehand in such a way as to promote binding with said first biological entities at said first electrodes (MIJ).

33. A method as in claim 32, wherein the immobilizing step comprises the single steps of:
a. introducing a suspension of said first biological entities into said chamber compassing said liquid or semi-liquid environment (L);
b. trapping and levitating said first biological entities within dielectrophoretic potential cages (S1, DEP) generated between selected first electrodes (LIJ) and said second electrode (M2);
c. selectively directing said dielectrophoretic cages (S1), with said first biological entities trapped within them, toward selected first electrodes (MIJ);
d. moving said cages (S1) in such a way as to bring about said binding between said first biological entities and said selected first electrodes (MIJ) and consequently immobilizing said first biological entities on said electrodes, according to a predetermined patterning sequence.

34. A method as in claim 33, further comprising the step of concentrating said first biological entities at selected first electrodes (MIJ) by bringing together and fusing two or more said dielectrophoretic cages (S1) containing one or more said first biological entities trapped within them.

35. A method as in claim 32, wherein said first biological entities are said known biological entities, and said second biological entities are said unknown biological entities, further comprising the steps of:
e. introducing a suspension of populations of second biological entities, conceivably of two or more different types, into said chamber;
f. concentrating at least one first part of the population of said second biological entities by attracting them into a dielectrophoretic cage (S1) generated between said electrodes (LIJ,M2);
g. moving said at least one first part of the population of said second biological entities and causing it to interact with at least part of a population of said known first biological entities immobilized on said surface at a selected first electrode (LIJ);
h. sensing any binding activity between at least one part of the population of unknown second biological entities and at least one part of the population of said known first biological entities immobilized on the first electrodes (LIJ).

36. A method as in claim 35, wherein said binding activity is verified by seeking to separate said populations of first and/or second biological entities one from another and/or from said first electrodes dielectrophoretically, trapping them within dielectrophoretic cages (S1) and distancing the cages from selected first electrodes (LIJ).

37. A method as in claim 36, wherein said binding activity is sensed by means of optical type sensors either located externally of said chamber or integrated into said array of first electrodes (LIJ).

38. A method as in claim 36, wherein said binding activity is sensed by means of capacitive type sensors.

39. A method as in claim 37, comprising a step of immobilizing said unknown second biological entities on microbeads having predetermined physical and chemical characteristics, such as will increase the capacitive or optical detectability of said binding activity.

40. A method as in claim 38, comprising a step of immobilizing said unknown second biological entities on microbeads having predetermined physical and chemical characteristics, such as will increase the capacitive or optical detectability of said binding activity.

41. A method as in claim 37, wherein use is made of optical sensors integrated into said array of first electrodes (LIJ), comprising the steps of: treating said second biological entities that will be bound to said first biological entities immobilized on the first electrodes (LIJ), with a substrate including fluorophore groups; exciting said fluorophores associated with the unknown first biological entities by exposing them to light emitted at a first wavelength (UV); sensing the emission of fluorescence at a second wavelength (LIG) different to the first by means of said integrated optical sensors in such a way as to determine the presence of the second biological entities bound to the first in close proximity to each first electrode (LIJ).

42. A method as in claim 32, wherein said first biological entities are said unknown biological entities, and said second biological entities are said known biological entities, further comprising the steps of:
i. immobilizing populations of second biological entities, conceivably of two or more different types, on microsupports having predetermined physical and chemical characteristics, conceivably different one to another;
l. introducing microsupports of at least one first type carrying said immobilized known second biological entities, into said chamber, and trapping them in dielectrophoretic cages (S1);
m. causing said microsupports trapped in said dielectrophoretic cages (S1) to interact with said surface consisting in said array of first electrodes (LIJ) occupied by immobilized populations of said unknown first biological entities conceivably different one from another;
n. verifying the force of any binding that occurs by seeking to separate said microsupports from said surface dielectrophoretically, trapping the microsupports within dielectrophoretic cages (S1) and distancing the cages from selected first electrodes (LIJ);
p. sensing a possible presence of the microsupports where binding occurs with said selected first electrodes (LIJ) to determine whether or not said binding is still occurring.

43. A method as in claim 42, wherein said microsupports are selected from a group including microbeads of synthetic material, cells and liposomes.

44. A method as in claim 43, wherein the microsupports are microbeads of at least two types distinguishable one from another on the basis of one or more physical parameters including dielectric constant, colour, transparency or fluorescence, further comprising the step of identifying the microsupport before implementing steps (n) and (p).

45. A method as in claim 42, wherein the step of causing interaction (m) is effected by shifting the dielectrophoretic cages (S1) toward the surface.

46. A method as in claim 42, wherein the step of causing interaction (m) is effected by eliminating the dielectrophoretic cages (S1) and causing the microsupports to precipitate onto the surface.

47. A method as in claim 42, wherein the step of causing interaction (m) is effected by changing the excitation frequency of said electrodes (LIJ) so as to generate a positive dielectrophoretic force (pDEP) such as will repel the microsupports from the respective dielectrophoretic cages (S1) and thus direct them into contact with the surface.

48. A method as in claim 42, wherein the step of verifying binding force (n) dielectrophoretically is effected by distancing the dielectrophoretic cages from the surface.

49. A method as in claim 46, wherein the step of verifying binding force (n) dielectrophoretically is effected by reactivating the dielectrophoretic cages (S1) to raise the microsupports from the surface.

50. A method as in claim 47, wherein the step of verifying binding force (n) dielectrophoretically is effected by restoring the initial excitation frequency so as to attract the microsupports toward the dielectrophoretic cages (S1).

51. A method as in claim 42, wherein the step of verifying binding force (n) dielectrophoretically is replaced with a verification step (n′) effected by exposing the microsupports to a flow of buffer solution directed through said chamber.

52. A method as in claim 42, wherein the step of sensing the presence of the microsupport (p) in the position of a selected electrode (LIJ) is effected utilizing a capacitive sensor associated with the electrode (LIJ).

53. A method as in claim 42, wherein the step of sensing the presence of the microsupport (p) at the site of a selected electrode (LIJ) is effected utilizing an optical sensor associated with the electrode (LIJ).

54. A method as in claim 53, wherein said optical sensor detects radiation emitted at a first frequency (LIG) from fluorophore groups associated with said microsupport, excited by radiation emitted at a second frequency (UV) not detectable by said optical sensor.

55. A method as in claim 53, wherein said optical sensor detects the variation in incident radiation accompanying the absorption or reflection by said microsupport of a measure of radiation originating externally to said test chamber.

56. A method as in claim 42, wherein the presence of said microsupport is sensed by an optical sensor located externally to said test chamber.

57. A device for carrying out molecular biological analyses performed with the aid of movable dielectrophoretic cages (S1, DEP) as claimed in the method according to anyone of the foregoing claims, comprising a surface afforded by an array (M1) of first electrodes (LIJ) selectively energizable and addressable at least in part and arranged on an insulating support (O1); at least one second electrode (M2) positioned opposite and facing at least a part of said array (M1) of first electrodes (LIJ); and a spacer serving to distance the first electrodes (LIJ) from said at least one second electrode (M2) in such a way that said second electrode, said spacer and said array (M1) of first electrodes combine to establish a test chamber encompassing a liquid or semi-liquid environment (L); and further comprising integrated optical sensors located beneath or in close proximity to at least one of said first electrodes (LIJ); characterized in that said integrated optical sensors consist in junction photodiodes (CPH) located at a given depth (DEP) from a surface of a semiconductor substrate (C) such as to render them substantially insensitive to radiation of a first predetermined wavelength (UV) and sensitive to radiation of a second predetermined wavelength.
